(19) United States
(12) Patent Application Publication
(10) Pub. No.: US 2026/0130739 A1
(43) Pub. Date: May 14, 2026

(54) JOINT LOCKING MECHANISM FOR HOLDING-ARMS OF MEDICAL SURGICAL DEVICES

(71) Applicant: KARL STORZ SE & Co. KG, Tuttlingen (DE)

(72) Inventors: Michael EGLE, Tuttlingen (DE); Pascal HENI, Tuttlingen (DE)

(21) Appl. No.: 19/101,974

(22) PCT Filed: Aug. 9, 2023
(86) PCT No.: PCT/EP2023/071996

§ 371 Date: Feb. 7, 2025

(30) Foreign Application Priority Data
Aug. 9, 2022 (DE) 10 2022 119 958.4

(51) Int. Cl. A61B 90/50

(52) U.S. Cl. CPC A61B 90/50; A61B 2090/508

(57) ABSTRACT

A joint locking mechanism (1) for holding arms of medical surgical devices with a gear (3) arranged on a pivot axis (2) of a joint and a spring-loaded roller (4, 5) which is in direct operative connection with the gear (3). The joint locking mechanism (1) holds arms of medical surgical devices, which ensures reduced wear on the roller (4, 5) and/or the gear (3) while maintaining the same positioning capability of the joint. The two spring-loaded rollers (4, 5) are in direct operative connection with at least one gear (3), such that in each latched position of the joint, at least one roller (4 or 5) always engages in a latching manner in a tooth gap (12) between two adjacent teeth (13) of at least one gear (3).

### CROSS REFERENCE TO RELATED APPLICATIONS

This application is a United States National Phase Application of International Application PCT/EP2023/071996, filed Aug. 9, 2023, and claims the benefit of priority under 35 U.S.C. § 119 of German Application 10 2022 119 958.4, filed Aug. 9, 2022, the entire contents of which are incorporated herein by reference.

### TECHNICAL FIELD

The invention relates to a joint locking mechanism for holding arms of medical surgical devices, comprising a gear arranged on a pivot axis of a joint and a spring-loaded roller which is in direct operative connection with the gear.

### BACKGROUND

Holding arms for medical surgical devices, such as surgical robots or holding stands, are known in a wide variety of designs from practice. In order to be able to position the holding arms in the required position of pivoting in space, the joints of the holding arms are equipped with joint locking mechanisms, by means of which the holding arms can be fixed in the currently required position.

From the prior art, a joint locking mechanism comprising a gear arranged on a pivot axis of a joint and a spring-loaded roller which is in direct operative connection with the gear is known. To lock the joint, the spring-loaded roller is pressed into a tooth gap between two teeth of the gear and thus prevents further pivoting of the holding arm. In order to be able to pivot the joint designed in this way in the smallest possible latching steps, the gear arranged on the pivot axis of the joint has as many teeth as possible and thus also as many tooth gaps as possible, the tooth gaps being provided for locking purposes. The more teeth a gear has, the narrower and more pointed the tooth tips of the teeth are.

The more tooth gaps there are on a gear, the narrower each tooth gap and the smaller the radius of the roller used for locking, which roller is to be accommodated in the tooth gap.

A joint locking mechanism designed in this way has certainly proven itself in practice, but it has been found that not only are the small rollers subjected to a high level of wear due to the contact pressure generated by the spring loading and the rolling over the sharp teeth or tooth tips, but also the teeth or tooth tips of the gear are subjected to a high level of wear, and this can lead to reduced locking performance of the joint.

Proceeding therefrom, the invention addresses the problem of designing a joint locking mechanism for holding arms of medical surgical devices in such a way that it ensures reduced wear on the roller and/or gear while maintaining the same positioning capability of the joint.

### SUMMARY

The solution to this problem is characterized according to the invention by two spring-loaded rollers, which are in direct operative connection with at least one gear such that, in each latched position of the joint, at least one roller always engages in a latching manner in a tooth gap between two adjacent teeth of at least one gear.

By using two spring-loaded rollers, it is possible to reduce the number of teeth of the at least one gear while maintaining the number of latched positions, which in turn also allows the radius of the two rollers to be increased. Increasing the roller diameters and dividing the contact pressure among two rollers significantly reduces the wear on the rollers as well as on the teeth or tooth tips of the at least one gear.

With a first embodiment of the invention it is proposed that the two rollers are tiltably mounted at the end of a pivot lever spring-loaded by means of at least one spring element and are in direct operative connection with a single gear, wherein the tilting plane in which the two rollers can be tilted relative to the gear is parallel to the gear. Tiltably mounting the two rollers facilitates the transition from one tooth gap to the next when the holding arm is pivoted into a new position.

To tiltably mount the two rollers, it is proposed according to the invention that the two rollers are arranged at the ends of a two-armed rocker lever, the rocker lever being pivotably mounted at the end of the pivot lever that is remote from the at least one spring element. By arranging the two rollers on a rocker lever, which in turn is pivotably mounted on the spring-loaded pivot lever, the contact force of the at least one spring element is applied evenly to both rollers.

Furthermore, the invention proposes that, in each latching position, one roller is arranged in a tooth gap of the gear, while the other roller is arranged on a tooth tip of the same gear. Due to this arrangement of the two rollers relative to one another and relative to the tooth gaps and tooth tips of the gear, it is possible that, with each rotation of the gear by only half a tooth gap distance, a roller, namely the one previously arranged on the tooth tip, engages in a tooth gap in a latching manner, while the roller previously arranged in the tooth gap is now arranged on the nearest tooth tip. This adjustment of the gear in half steps, measured by the tooth gap distance of the gear, enables the same number of latched positions of the joint as when using a gear with twice as many teeth.

According to a second embodiment of the invention, it is proposed that the two rollers are tiltably mounted at the end of a pivot lever spring-loaded by means of at least one spring element and each of the two rollers is in direct operative connection with a separate gear, the two rollers being tiltable relative to the two gears in two parallel tilting planes, which are both parallel to the two gears. In this embodiment, the contact pressure exerted on the pivot lever and thus on the two rollers by means of the at least one spring element is evenly transferred to the gears associated with the respective rollers, in order to achieve effective locking of the joint.

Furthermore, the invention proposes that, when two gears are used, the two identical gears are arranged parallel to one another on the pivot axis of the joint in such a way that each tooth of one gear is arranged centrally with respect to a tooth gap of the other gear.

To tiltably mount the two rollers, it is proposed according to the invention that the two rollers are arranged at the ends of a two-armed rocker lever, the rocker lever being pivotably mounted at the end of the pivot lever that is remote from the at least one spring element. By arranging the two rollers on a rocker lever, which in turn is pivotably mounted on the spring-loaded pivot lever, the contact force of the at least one spring element is applied evenly to both rollers.

This arrangement of the two gears relative to one another likewise allows latching in half steps, measured by the tooth gap distance of the individual gears; this enables the same number of latched positions of the joint as when using a gear with twice as many teeth.

In this arrangement of the two gears in relation to one another according to the invention, in each latching position one roller is arranged in a tooth gap of one gear while the other roller is arranged on a tooth tip of the other gear.

In order to avoid play between the roller arranged in a tooth gap and the particular tooth gap in the latching positions of the joint locking mechanism, which play would impair the exact positioning of the holding arm, the invention proposes that each roller, when arranged in a tooth gap, contacts only a point on each of the tooth flanks which are located on both sides of the particular tooth gap. The contact of the rollers at only a point on each of the tooth flanks guarantees an exact and play-free positioning of the particular roller in the associated tooth gap.

According to the invention, the contact regions of the tooth flanks at which a roller rests at a point on each of the tooth flanks are configured as straight lines in order to ensure, for secure latching of the joint, defined introduction of the contact force of the roller into the tooth flanks tangentially contacting the roller.

Finally, the invention proposes that the tooth base of each tooth gap between two adjacent teeth has the shape of a circular arc such that the radius of each tooth base of the at least one gear is smaller than the radius of each roller. By forming the two different radii, it is easy from a manufacturing perspective to achieve contact of the rollers at only a point on each of the tooth flanks.

The various features of novelty which characterize the invention are pointed out with particularity in the claims annexed to and forming a part of this disclosure. For a better understanding of the invention, its operating advantages and specific objects attained by its uses, reference is made to the accompanying drawings and descriptive matter in which preferred embodiments of the invention are illustrated.

### BRIEF DESCRIPTION OF THE DRAWINGS

In the drawings:

FIG. 1 is a schematic front view of a first embodiment of a joint locking mechanism for holding arms according to the invention;

FIG. 2 is a schematic front view of a second embodiment of a joint locking mechanism for holding arms according to the invention; and

FIG. 3 is an enlarged schematic representation of detail III according to FIG. 1.

### DESCRIPTION OF PREFERRED EMBODIMENTS

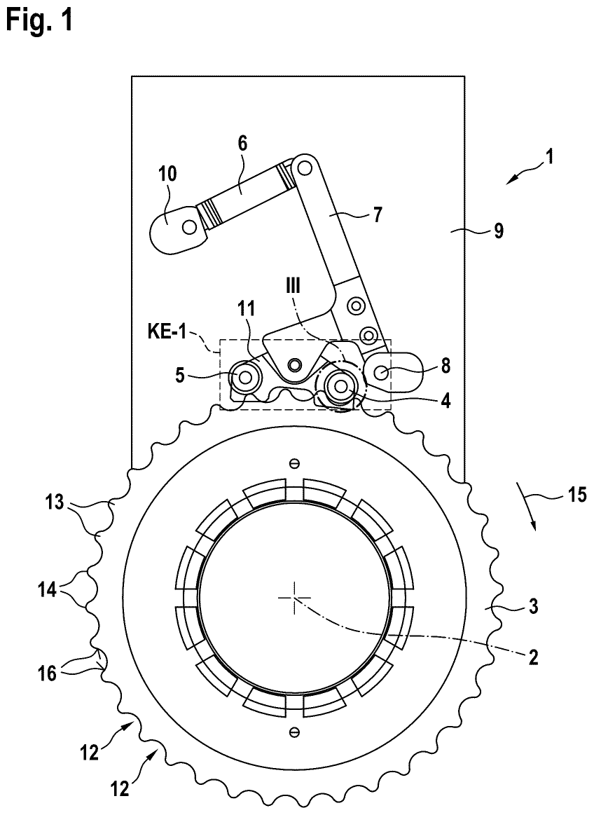

Referring to the drawings, FIG. 1 shows a joint locking mechanism 1 for holding arms of medical surgical devices, such as surgical robots or holding stands.

The holding arm, which can be pivoted about a pivot axis 2, is not shown in the figures, since the structure and design of the joint locking mechanism 1 is independent of the design of the holding arm.

In order to be able to position the holding arms in the required position of pivoting in space, the joints of the holding arms are equipped with at least one joint locking mechanism 1, by means of which the particular holding arm can be fixed in the currently required position.

The joint locking mechanism 1 shown in FIG. 1 essentially consists of a gear 3 arranged on the pivot axis 2 of a joint and two spring-loaded rollers 4 and 5 which are in direct operative connection with the gear 3. For this purpose, the two rollers 4, 5 are tiltably mounted at the end of a pivot lever 7 spring-loaded by means of at least one spring element 6.

The pivot lever 7 is in turn mounted on a joint housing 9 so as to be pivotable about a pivot axis 8. The at least one spring element 6 is mounted, at one end, on the pivot lever 7 and, at the other end, on a support 10 on the joint housing 9.

As can also be seen from FIG. 1, the two rollers 4 and 5 are arranged at the ends of a two-armed rocker lever 11, the rocker lever 11 being pivotably mounted at the end of the pivot lever 7 that is remote from the at least one spring element 6. With this arrangement of the rollers 4 and 5 on the rocker lever 11, the tilting plane KE-1 in which the two rollers 4 and 5 can be tilted relative to the gear 3 is parallel to the gear 3.

The two rollers 4 and 5 are arranged relative to the gear 3 on the rocker lever 11 such that, in each latching position, one roller 4 or 5 is arranged in a tooth gap 12 between two adjacent teeth 13 of the gear 3, while the other roller 5 or 4 is arranged on a tooth tip 14 of a tooth 13 of the same gear 3.

The joint locking mechanism 1 shown in FIG. 1 works as follows:
When the holding arm of the medical surgical device is pivoted about the pivot axis 2, the gear 3 arranged on the pivot axis 2 is inevitably also rotated about the pivot axis 2 to the same extent as the holding arm.

In the latched position shown in FIG. 1, the pivot lever 7 presses the two rollers 4 and 5, which are tiltably mounted on the rocker lever 11, downwards towards the gear 3 with the spring force of the spring element 6, such that the roller 4 accommodation is arranged in a tooth gap 12 between two adjacent teeth 13 of the gear 3, while the other roller 5 is arranged on a tooth tip 14 of a tooth 13 of the same gear 3.

If the gear 3 is now rotated clockwise in the direction of the arrow 15 by the holding arm, this causes the roller 4 arranged in the tooth gap 12 to roll up the tooth flank 16 of the adjacent tooth 13 to the tooth tip 14, while the roller 5 arranged on the tooth tip 14 rolls down the nearest tooth flank 16 into the tooth gap 12.

Due to this arrangement of the two rollers 4 and 5 relative to one another and relative to the tooth gaps 12 and tooth tips 14 of the gear 3, it is possible that, with each rotation of the gear 3 by only half a tooth gap distance, a roller 4 or 5, namely the one previously arranged on the tooth tip 14, engages in a tooth gap 12 in a latching manner, while the roller 4 or 5 previously arranged in the tooth gap 12 is now arranged on the nearest tooth tip 14. This adjustment of the gear 3 in half steps, measured by the tooth gap distance of the gear 3, enables the same number of latched positions of the joint as when using a gear with twice as many teeth but only one roller for latching in a tooth gap 12. Only the mounting of the rollers 4 and 5 on the rocker lever 11, which can be tilted relative to the pivot lever 7, enables this latching of the rollers 4 and 5 at distances which are half the tooth gap distance.

The spring-loaded pivot lever 7 ensures that the rollers 4 and 5 are always in direct operative connection with the gear 3 and that the particular roller 4 or 5 arranged in a tooth gap 12 fixes the gear 3 and thus the holding arm in the particular position.

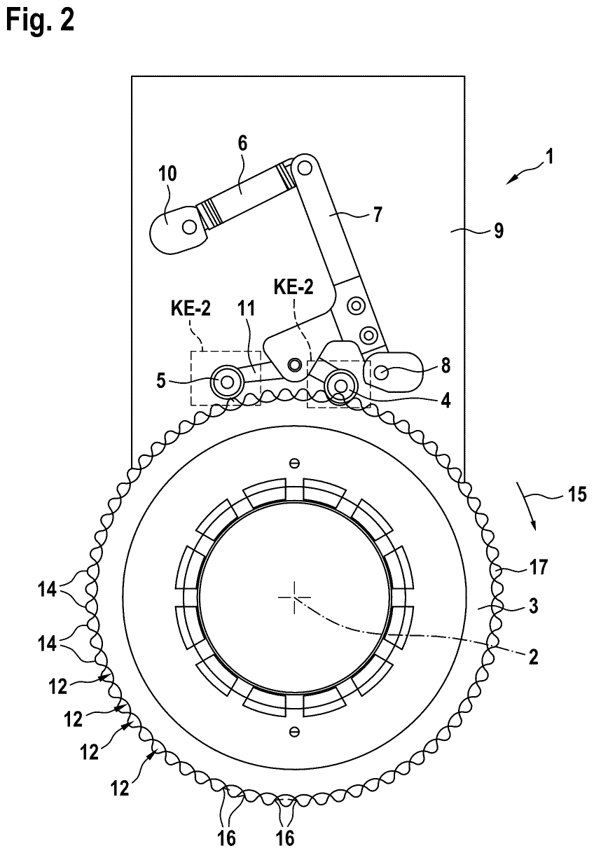

The second embodiment of the joint locking mechanism 1 shown in FIG. 2 differs from the joint locking mechanism 1 shown in FIG. 1 and described above in that two identical gears 3 and 17 are now arranged parallel to one another on the pivot axis 2 of the joint.

As can be seen from FIG. 2, the two gears 3 and 17 are arranged relative to one another in such a way that each tooth 13 of one gear 3 or 17 is arranged centrally with respect to a tooth gap 12 of the other gear 17 or 3.

In this embodiment of the joint locking mechanism 1, the two rollers 4 and 5 are tiltably mounted at the end of the pivot lever 7 spring-loaded by means of at least one spring element 6, such that each of the two rollers 4 and 5 is in direct operative connection with a separate gear 3, 17.

The spring-loaded pivot lever 7 is in turn mounted on the joint housing 9 so as to be pivotable about a pivot axis 8, and the at least one spring element 6 is mounted, at one end, on the pivot lever 7 and, at the other end, on a support 10 on the joint housing 9.

As can also be seen from FIG. 2, the two rollers 4 and 5 are arranged at the ends of a two-armed rocker lever 11, the rocker lever 11 being pivotably mounted at the end of the pivot lever 7 that is remote from the at least one spring element 6.

With this arrangement of the rollers 4 and 5 on the rocker lever 11, the two rollers 4 and 5 can be tilted in two parallel tilting planes KE-2 relative to the two gears 3 and 17, the two tilting planes KE-2 being parallel to the two gears 3 and 17.

The two rollers 4 and 5 are arranged relative to the gears 3 and 17 on the rocker lever 11 such that, in each latching position, one roller 4 or 5 is arranged in a tooth gap 12 between two adjacent teeth 13 of one gear 3 or 17, while the other roller 5 or 4 is arranged on a tooth tip 14 of a tooth 13 of the other gear 17 or 3.

The joint locking mechanism 1 shown in FIG. 2 works as follows:

When the holding arm of the medical surgical device is pivoted about the pivot axis 2, the two gears 3 and 17 arranged on the pivot axis 2 are inevitably also rotated about the pivot axis 2 to the same extent as the holding arm.

In the latched position shown in FIG. 2, the pivot lever 7 presses the two rollers 4 and 5, which are tiltably mounted on the rocker lever 11, downwards towards the two gears 3 and 17 with the spring force of the spring element 6, such that the roller 4 accommodation is arranged in a tooth gap 12 between two adjacent teeth 13 of the gear 3, while the other roller 5 is arranged on a tooth tip 14 of a tooth 13 of the other gear 17.

If the two gears 3 and 17 are now rotated clockwise in the direction of the arrow 15 by the holding arm, this causes the roller 4 arranged in the tooth gap 12 of the gear 3 to roll up the tooth flank 16 of the adjacent tooth 13 to the tooth tip 14, while the roller 5 arranged on the tooth tip 14 of the gear 17 rolls down the nearest tooth flank 16 into the tooth gap 12.

Due to this arrangement of the two rollers 4 and 5 relative to one another and relative to the tooth gaps 12 and tooth tips 14 of the two gears 3 and 17, it is possible that, with each rotation of the gears 3 and 17 by only half a tooth gap distance, a roller 4 or 5, namely the one previously arranged on the tooth tip 14 of one gear 3 or 17, engages in a tooth gap 12 in a latching manner, while the roller 4 or 5 previously arranged in the tooth gap 12 of the gear 17 or 3 is now arranged on the nearest tooth tip 14.

This adjustment of the gear 3 in half steps, measured by the tooth gap distance of the gear 3, enables the same number of latched positions of the joint as when using a gear with twice as many teeth but only one roller for latching in a tooth gap 12. Only the mounting of the rollers 4 and 5 on the rocker lever 11, which can be tilted relative to the pivot lever 7, enables this latching of the rollers 4 and 5 at distances which are half the tooth gap distance.

The spring-loaded pivot lever 7 ensures that the rollers 4 and 5 are always in direct operative connection with the two gears 3 and 17 and that the particular roller 4 or 5 arranged in a tooth gap 12 fixes the gear 3 or 17 and thus the holding arm in the particular position.

The two embodiments for designing a joint locking mechanism 1 which are shown in FIGS. 1 and 2 are characterized in that, due to the two rollers 4 and 5 used, which enable latching at distances which are half the tooth gap distance, the individual gears 3 and 17 can have relatively wide tooth gaps and the rollers 4 and 5 can also have a correspondingly large radius, so that the wear on the rollers 4 and 5 and on the tooth tips 14 of the gears 3 and 17 is significantly reduced in comparison with the joint locking mechanisms 1 known from the prior art.

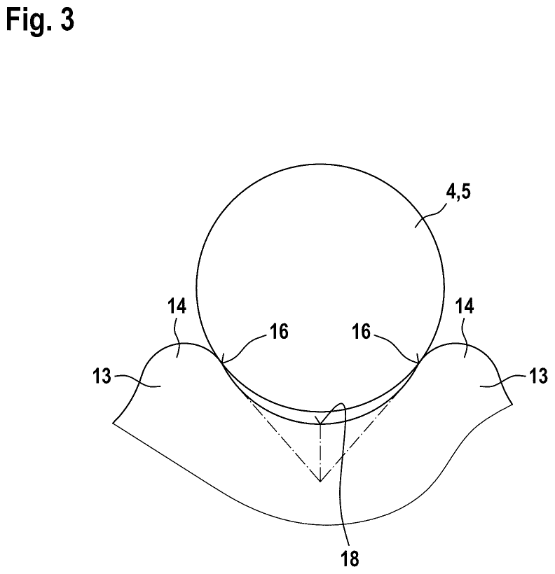

In order to avoid play between the roller 4 or 5 arranged in a tooth gap 12 and the particular tooth gap 12 in the latching positions of the joint locking mechanism 1, which play would impair the exact positioning of the holding arm, each roller 4 or 5, when arranged in a tooth gap 12 of a gear 3 or 17, contacts only a point on each of the tooth flanks 16 which are located on both sides of the particular tooth gap 12, as shown in FIG. 3.

The contact of the rollers 4 or 5 at only a point on each of the tooth flanks 16 guarantees an exact and play-free positioning of the particular roller 4 or 5 in the associated tooth gap 12.

The contact regions of the tooth flanks 16 at which a roller 4 or 5 rests at a point on each of the respective tooth flanks 16 are advantageously configured as a straight line in order to ensure, for secure latching of the joint, defined introduction of the contact force of the roller 4 or 5 into the tooth flanks 16 tangentially contacting the roller 4 or 5.

As can also be seen from FIG. 3, the tooth base 18 of each tooth gap 12 between two adjacent teeth 13 has the shape of a circular arc such that the radius of each tooth base 18 of the gears 3 and 17 is smaller than the radius of each roller 4 or 5. By forming the two different radii, it is easy from a manufacturing perspective to achieve contact of the rollers 4 or 5 at only a point on each of the tooth flanks 16.

The invention relates to a joint locking mechanism 1 for holding arms of medical surgical devices, comprising a gear 3 arranged on a pivot axis 2 of a joint and a spring-loaded roller 4, 5 which is in direct operative connection with the gear 3. A joint locking mechanism 1 for holding arms of medical surgical devices, which ensures reduced wear on the roller 4, 5 and/or gear 3 while maintaining the same positioning capability of the joint, is characterized according to the invention by two spring-loaded rollers 4, 5, which are in direct operative connection with at least one gear 3 such that, in each latched position of the joint, at least one roller 4 or 5 always engages in a latching manner in a tooth gap 12 between two adjacent teeth 13 of at least one gear 3. The drawings, the description, and the claims contain numerous features in combination. It goes without saying that the above-mentioned features can be used not only in the combination specified in each case, but also in other combinations or on their own, without departing from the scope of the present invention.

While specific embodiments of the invention have been shown and described in detail to illustrate the application of the principles of the invention, it will be understood that the invention may be embodied otherwise without departing from such principles.

### LIST OF REFERENCE SIGNS

1 Joint locking mechanism
2 Pivot axis
3 Gear
4 Roller
5 Roller
6 Spring element
7 Pivot lever
8 Pivot axis
9 Joint housing
10 Support
11 Rocker lever
12 Tooth gap
13 Tooth
14 Tooth tip
15 Arrow (clockwise)
16 Tooth flank
17 Gear
18 Tooth base
KE-1 Tilting plane
KE-2 Tilting plane

1. A joint locking mechanism for holding arms of medical surgical devices, the join locking mechanism comprising:
a gear arranged on a pivot axis of a joint; and
two spring-loaded rollers which are in direct operative connection with the gear such that, in each latched position of the joint, at least one roller always engages in a latching manner in a tooth gap between two adjacent teeth of the gear.

2. The joint locking mechanism according to claim 1, wherein the two one rollers are tiltably mounted at the end of a pivot lever spring-loaded by means of at least one spring element and are in direct operative connection with the gear, wherein a tilting plane in which the two rollers can be tilted relative to the gear is parallel to the gear.

3. The joint locking mechanism according to claim 2, wherein the two rollers are arranged at the ends of a two-armed rocker lever, the rocker lever being pivotably mounted at the end of the pivot lever that is remote from the at least one spring element.

4. The joint locking mechanism according to claim 2, wherein in each latching position, one roller is arranged in a tooth gap of the gear, while the other roller is arranged on a tooth tip of the same gear.

5. The joint locking mechanism according to claim 1, wherein the gear comprises a two gear configuration and the two rollers are tiltably mounted at the end of a pivot lever spring-loaded by means of at least one spring element and each of the two rollers is in direct operative connection with a separate gear of the two gear configuration, the two rollers being tiltable relative to the two gears in two parallel tilting planes (KE-2), which are both parallel to the two gears.

6. The joint locking mechanism according to claim 5, wherein the two gear configuration comprises two identical gears that are arranged parallel to one another on the pivot axis of the joint in such that each tooth of one gear is arranged centrally with respect to a tooth gap of the other gear.

7. The joint locking mechanism according to claim 5, wherein the two rollers are arranged at the ends of a two-armed rocker lever, the rocker lever being pivotably mounted at the end of the pivot lever that is remote from the at least one spring element.

8. The joint locking mechanism according to claim 5, wherein in each latching position, one roller is arranged in a tooth gap of one gear of the two gear configuration, while the other roller is arranged on a tooth tip of the other gear of the two gear configuration.

9. The joint locking mechanism according to claim 1, wherein each roller, when arranged in a tooth gap, contacts only a point on each of tooth flanks which are located on both sides of the tooth gap.

10. The joint locking mechanism according to claim 9, wherein contact regions of the tooth flanks at which a roller contacts only a point on each of the tooth flanks which are located on both sides of the tooth gap are configured as a straight line.

11. The joint locking mechanism according to claim 1, wherein the tooth base of each tooth gap between two adjacent teeth has the shape of a circular arc such that a radius of each tooth base of the at least one gear is smaller than the radius of each roller.

12. A joint locking mechanism for holding arms of medical surgical devices, the join locking mechanism comprising:
a gear configuration arranged on a pivot axis of a joint; and
a spring loaded roller configuration comprising two spring-loaded rollers, which roller configuration is in direct operative connection with the gear configuration to provide a latched position of the joint with at least one roller of the roller configuration always in latching engagement with a tooth gap between two adjacent teeth of the gear configuration.

13. The joint locking mechanism according to claim 12, wherein the spring loaded roller configuration further comprises a pivot lever with the two rollers tiltably mounted at an end thereof via at least one spring element and with the two rollers in direct operative connection with a single gear of the gear configuration, wherein a tilting plane in which the two rollers are tiltable relative to the single gear is parallel to the single gear.

14. The joint locking mechanism according to claim 13, wherein the spring loaded roller configuration further comprises a two-armed rocker lever wherein the two rollers are arranged at the ends of the two-armed rocker lever and the rocker lever is pivotably mounted at an end of the pivot lever that is remote from the at least one spring element.

15. The joint locking mechanism according to claim 13, wherein in each latching position, one roller is arranged in a tooth gap of the single gear, while the other roller is arranged on a tooth tip of the single gear.

16. The joint locking mechanism according to claim 12, wherein the gear configuration comprises two gears and the two rollers and the spring loaded roller configuration further comprises a pivot lever spring-loaded by means of at least one spring element such that the two gears are tiltably mounted at the end of pivot lever and each of the two rollers is in direct operative connection with a separate gear of the gear configuration, the two rollers being tiltable relative to the two gears in two parallel tilting planes, which are both parallel to the two gears.

17. The joint locking mechanism according to claim 16, wherein the two gears comprises two identical gears that are arranged parallel to one another on the pivot axis of the joint in such that each tooth of one gear is arranged centrally with respect to a tooth gap of the other gear.

18. The joint locking mechanism according to claim 16, wherein the spring loaded roller configuration further comprises a two-armed rocker lever and wherein the two rollers are arranged at the ends of the two-armed rocker lever, the rocker lever being pivotably mounted at the end of the pivot lever that is remote from the at least one spring element.

19. The joint locking mechanism according to claim 16, wherein in each latching position, one roller is arranged in a tooth gap of one gear of the gear configuration, with the other roller arranged on a tooth tip of the other gear of the gear configuration.
